(19) United States
(12) Patent Application Publication
(10) Pub. No.: US 2009/0012472 A1
(43) Pub. Date: Jan. 8, 2009

(54) Medical Device with Cannula Inserter

(75) Inventors: Thorkild Ahm, Allerød (DK); Claude Teisen-Simony, Frederiksberg (DK); Simon Rorvig, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, Bagsvaerd (DK)

(21) Appl. No.: 11/662,905

(22) PCT Filed: Sep. 22, 2005
(86) PCT No.: PCT/EP2005/054758

§ 371 Date: Apr. 11, 2008

(30) Foreign Application Priority Data
Sep. 22, 2004 (DK) PA 2004 01434
Dec. 6, 2004 (DK) PA 2004 01894
Jan. 24, 2005 (DK) PA 2005 00115

(51) Int. Cl. A61M 5/20

(52) U.S. Cl. 604/138

(57) ABSTRACT

A medical device is provided comprising a cannula (771, 861) having a distal end portion adapted to be arranged through the skin of the subject and having a distal opening, and a needle (761, 861) arranged coaxially with and being axially moveable relative to the cannula, the needle comprising a pointed distal end, wherein the device is adapted for advancing the cannula with the distal end of the needle projecting there from through the dermis of the subject, and further advancing the cannula into the subcutis of the subject with the distal end of the cannula projecting relative to the needle.

The present invention generally relates to a device which is adapted for application to a skin surface of a subject and comprises a cannula in combination with an insertion needle, the insertion needle serving as an insertion aid for the cannula which typically is more flexible than the insertion needle. In embodiments of the invention the cannula may be replaced with a sensor.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a hollow infusion needle, as well as expelling means for expelling a drug out of the reservoir and through the skin of the subject via the hollow needle. Such devices are often termed infusion pumps.

Basically, infusion pumps can be divided into two classes. The first class comprises infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniences, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also know as a bleeding hole pump)), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference. U.S. Pat. No. 6,364,865 discloses a manually held infusion device allowing two vial-type containers to be connected and a pressure to be build up in one of the containers to thereby expel a drug contained in that container.

The disposable pumps generally comprises a skin-contacting mounting surface adapted for application to the skin of a subject by adhesive means, and with the infusion needle arranged such that in a situation of use it projects from the mounting surface to thereby penetrate the skin of the subject, whereby the place where the needle penetrates the skin is covered while the appliance is in use. The infusion needle may be arranged to permanently project from the mounting surface such that the needle is inserted simultaneously with the application of the infusion pump, this as disclosed in U.S. Pat. Nos. 2,605,765, 4,340,048 and in EP 1 177 802, or the needle may be supplied with the device in a retracted state, i.e. with the distal pointed end of the needle “hidden” inside the pump device, this allowing the user to place the pump device on the skin without the possibility of observing the needle, this as disclosed in U.S. Pat. Nos. 5,858,001 and 5,814,020.

As an alternative to a needle, a cannula in combination with an insertion needle which is withdrawn after insertion thereof may be used. Typically, the cannula is in the form of a relatively soft infusion cannula (e.g. a Teflon® cannula) and a there through arranged removable insertion needle. This type of cannula and needle arrangement is well known from so-called infusion sets, such infusion sets typically being used to provide an infusion site in combination with (durable) infusion pumps. However, recently a disposable pump has been disclosed comprising an insertable soft cannula in combination with an insertion needle. More specifically, WO 03/090509 shows a skin mountable drug delivery device comprising an initially concealed soft cannula through which an insertion needle is arranged. With the device mounted on a skin surface the cannula can be released and inserted angled through the skin, the pointed distal end of the insertion needle projecting from the distal end of the cannula. When the cannula is fully inserted the insertion needle is withdrawn.

For all of the above the following has to be considered. When a needle enters the subcutaneous tissue, the cutting edge will cause lesions to this tissue; cells are punctured, the fine blood vessels are damaged and nerve connections are cut, causing hemorrhage and trauma to the patient. Further, such hemorrhage triggers an immune reaction in the tissue, causing the chemical environment in the skin at the insertion site to change. This can influence the effect of the injected substance, which of course is undesirable. This is especially an issue for obliquely inserted soft cannulas as these are normally inserted with a long travel to reach the desired depth of insertion in the subcutaneous space.

### DISCLOSURE OF THE INVENTION

Having regard to the above-identified problems, it is an object of the present invention to provide a skin mountable medical device or system as well as components therefore, which allow such a device or system to be used in a convenient and cost-effective manner, yet allowing safe and reliable treatment of a medical condition.

In the disclosure of the present invention, embodiments and aspects will be described which will address one or more of the above objects or which will address objects apparent from the below disclosure as well as from the description of exemplary embodiments.

Thus, a medical device is provided comprising a housing adapted for application towards the skin of a subject, a cannula having a distal end portion adapted to be arranged through the skin of the subject and having a distal opening, and an insertion needle (in the following also denoted as a needle for short) arranged coaxially with and being axially moveable relative to the cannula, the needle comprising a pointed distal end. The medical device is transformable between (1) a first state in which the cannula and the needle are retracted within the housing, (2) a second state in which the cannula and the needle are extended relative to the lower surface with the distal end of the needle projecting relative to the distal opening of the cannula thereby allowing the cannula to be introduced through the skin of the subject, (3) a third state in which the distal end of the needle is positioned short of the distal opening, the cannula not being fully extended relative to the housing, (4) a fourth state in which the cannula is fully extended relative to the housing, and optionally (5) a fifth state in which the needle is retracted from the portion of the cannula extending from the housing.

The cannula will typically be in the form of a flexible, relatively soft polymeric tube having a relatively blunt distal end (often designated a catheter or soft catheter), with the needle typically being formed from medical grade stainless steel providing the pointed distal end, however, the needle may also be formed from a polymeric material.

The term “housing” merely denotes a supporting structure for supporting the different elements as described. The housing may be a traditional partially or fully closed structure, however, it may also be in the form of an open structure, e.g. a platform.

Corresponding to a first aspect, the needle is arranged within the cannula such that in the fourth state the distal end of the needle can be positioned within the cannula short of the distal opening. The above arrangement allows the upper leathery layer of the skin to be penetrated with the insertion needle projecting from the cannula. Thereafter the distal ends of the cannula and the needle “shift positions”, e.g. the needle stops and the cannula continues the insertion until the distal end is a short distance in front of the needle end, e.g. 1-5 mm. After this the cannula and the needle together continue the insertion through the relatively soft subcutaneous tissue, the needle providing directional guidance as well as support against kinking, until the cannula is fully inserted. As appears, the combined cannula and needle assembly has a blunt tip when penetrating the sub-cutis thereby causing reduced damage to the subcutaneous tissue. Compared with traditional infusion sets in which the needle penetrates the dermis as well as sub-cutis, less damage can be expected. Once the cannula is fully inserted, the needle is retracted.

To provide the relative motions between the cannula, the needle and the housing, an exemplary embodiment comprises an inserter assembly for moving the cannula and the insertion needle between the different states as defined above. The inserter assembly comprises the cannula, an inserter for moving the cannula, the needle, and a needle holder attached to the needle. The inserter assembly has (a) an initial state in which the needle holder is locked to the inserter in an initial position with the distal end of the needle projecting from the distal opening of the cannula, (b) an intermediate state in which the needle holder is locked to the inserter in an intermediate position with the distal end of the needle positioned within the cannula short of the distal opening, and optionally (c) a retracted state in which the needle is retracted from the portion of the cannula extending from the housing. This arrangement allows the inserter to function as the primary vehicle for moving the cannula and the needle, the “shift” between the initial and the intermediate state allowing the relative movement between the cannula and needle after the initial insertion through the outer layer of the skin.

More explicitly, the above arrangement provides a medical device wherein (i) the inserter assembly is transformable from the first to the second state with the inserter assembly in its initial state (e.g. the entire inserter assembly is moved forward), (ii) the inserter assembly is transformable from the second to the third state when the inserter is transformed from its initial to its intermediate state (e.g. the needle holder is released from its initial position whereby movement of the needle stops as the inserter and the cannula move forward, the needle holder thereafter being locked in its intermediate position after which it will be moved together with the cannula), (iii) the inserter assembly is transformable from the third to the fourth state with the inserter assembly in its intermediate state (e.g. the entire inserter assembly is moved forward with the needle locked in its intermediate state), and (iv) the inserter assembly is transformable from the fourth to the fifth state when the inserter is transformed from its intermediate to its retracted state (e.g. the needle is retracted from the cannula, either by retracting the needle holder or by retracting the inserter with the needle holder locked in place).

To prevent displacement of the inserted cannula, the fully extended cannula may be locked in place relative to the housing. For example, in case the inserter is left in place and only the needle is withdrawn, locking means (e.g. barbs or hooks) may be provided between the housing and the inserter. Alternatively, the cannula may be attached to a cannula which can be moved by the inserter from a retracted to a fully extended position, the cannula holder and the housing comprising cooperating fastening means for locking the cannula in its extended position, this allowing the inserter to be withdrawn after insertion.

To provide swift and minimally painful insertion, a user-releaseable actuator for actuating the inserter assembly from state 1-4 may be provided, i.e. until the cannula has been fully inserted. For example, the actuator may comprise a spring urging the inserter from an initial position to an extended position corresponding to the first respectively the fourth state. Further, a retractor for retracting the needle from its fully extended to a retracted position may be provided, e.g. a handle or a strip allowing the needle to be pulled back, or a further spring actuated mechanism. The latter may be coupled to a first actuator to provide fully automatic insertion corresponding to states 1-5.

In an exemplary embodiment the medical device comprises first and second housing portions coupled to each other, wherein the cannula in its extended position can be locked to the first housing portion. When the needle is retracted into the second housing portion, the second housing portion can be detached from the first housing portion with the needle being arranged there within. Preferably a lock is provided locking the needle safely within the second housing after use, this preventing unintended needle sticks. This concept may also be utilized in a medical device comprising a second housing with a “traditional” combination of a cannula and a needle, i.e. an arrangement in which the cannula and needle assembly is moved to their extended position with the needle projecting from the cannula.

The medical device may be provided with a flexible sheet member with a lower surface adapted to be arranged on a skin surface of a subject (e.g. comprising an adhesive), and an upper surface to which the first housing portion is arranged. In this way the cannula can be securely held in place after insertion.

After the cannula has been inserted it may be coupled to a desired fluid source, e.g. tubing to supply fluids or drugs from an IV bag or bottle. The medical device may also be provided as part of an assembly comprising a medical device as described above and a delivery device adapted to be coupled to the first housing portion. Such a delivery device comprising a reservoir adapted to contain a fluid drug, and an expelling assembly adapted for cooperation with the reservoir to expel fluid drug out of the reservoir and through the skin of the subject via the cannula when the delivery device has been coupled to the first housing portion after the cannula has been inserted and the second housing portion removed. As appears, in case fluid is supplied directly to the cannula, the needle has to be removed before attachment of the delivery device.

Alternatively, the needle may be hollow and comprise a proximal end, with the distal end of the needle being in sealed fluid communication with the interior of the cannula when the needle has been arranged in its retracted position. By this arrangement a fluid communication can be provided between the proximal end of the needle and the cannula. In this case a delivery device would supply drug to the cannula via the hollow needle.

Corresponding to a further aspect, the needle is hollow and arranged outside the cannula, this allowing for a smaller diameter cannula as it does not have to accommodate the needle. On the other hand the needle will have a larger diameter. Thus the needle may be arranged to be fully extended corresponding to the third state, i.e. the outer needle is only used to penetrate the uppermost layer of the skin and does not support the cannula during the further insertion in the subcutis.

Accordingly, in a further embodiment a medical device is provided comprising an inserter assembly for moving the cannula and the insertion needle between the different states. The inserter assembly comprises the cannula, the needle, an inserter attached to the needle, a cannula holder attached to the cannula and adapted for moving the cannula relative to the inserter and thereby the needle. The inserter assembly has (a) an initial state in which the cannula is positioned within the needle and with the distal end of the needle projecting relative to the distal opening of the cannula, and (b) an intermediate state in which the cannula holder has been moved to extend the cannula from the needle. The inserter assembly may have (c) a further extended state in which the cannula holder has been moved to further extend the cannula from the needle. Further, the inserter assembly has (d) a retracted and (e) an extended position.

More explicitly, the above arrangement can provide a medical device wherein (i) the inserter assembly is transformable from the first to the second state when the inserter assembly is moved from the retracted to the extended position with the inserter assembly in its initial state (i.e. the entire inserter assembly is moved forward), (ii) the inserter assembly is transformable from the second to the third state when the inserter is transformed from its initial to its intermediate state (i.e. the cannula is extended from the needle), and (iii) the inserter assembly is transformable from the third to the fourth state with the inserter assembly in its intermediate state (i.e. the cannula is further extended from the needle). The inserter assembly may further (iv) be transformable from the fourth to the fifth state when the inserter is transformed from its intermediate to its extended state and when the inserter assembly is moved from the extended to the retracted position (i.e. the cannula is fully extended from the needle and the entire inserter assembly is retracted).

As for the above described embodiment, the medical may comprise a flexible sheet member with a lower surface adapted to be arranged on a skin surface of a subject, and an upper surface to which the housing is arranged. Such a medical device may also be provided as part of an assembly further comprising a delivery device adapted to be coupled to the housing, the delivery device comprising a reservoir adapted to contain a fluid drug, and an expelling assembly adapted for cooperation with the reservoir to expel fluid drug out of the reservoir and through the skin of the subject via the cannula when the delivery device has been coupled to the first housing portion after the cannula has been inserted and the second housing portion removed. The assembly may also be provided as a unitary device.

In the above embodiments the subcutaneously introduced element has been in the form of a cannula suitable for drug delivery, however, the cannula may be replaced by a sensor and the delivery device may be in the form of an assembly adapted to transmit and/or process data acquired via the sensor, see for example U.S. Pat. No. 5,482,473 which is hereby incorporated by reference. A penetrating sensor may allow a body parameter to be sensed in the subcutaneous space, e.g. by using a needle formed sensor as discussed in the introduction, or by transporting fluid from the subcutaneous space to detection assembly by means of a conduit, this principle being known as micro-dialysis. An example of a penetrating needle-sensor and a corresponding process unit is shown in U.S. Pat. No. 6,809,653 (hereby incorporated by reference) which discloses a characteristic monitor system including a data receiving device, a transcutaneous needle sensor for producing signal indicative of a characteristic of a subject (e.g. a blood glucose value), and a processor device. The processor device includes a housing, a sensor connector, a processor, and in the shown embodiment a transmitter. In the shown embodiment the processor coupled to the sensor processes the signals from the sensor for transmission to the remotely located data receiving device, however, the processed data could also be shown directly on a display provided on the processor device. The data receiving device may be a characteristic monitor, a data receiver that provides data to another device, a wireless programmer for a medical device (e.g. a remote control), a medication delivery device (such as an infusion pump), or the like.

In a further aspect a method of inserting a cannula into the subcutaneous tissue of a subject is provided, comprising the steps of (a) providing a cannula having a distal end portion adapted to be arranged through the skin of the subject and having a distal opening, and a needle arranged coaxially with and being axially moveable relative to the cannula, the needle comprising a pointed distal end, (b) advancing the cannula with the distal end of the needle projecting there from through the dermis of the subject, and (c) further advancing the cannula into the sub-cutis of the subject with the distal end of the cannula projecting relative to the needle. During the advancement of the cannula into the sub-cutis the distal end of the needle may be arranged short of the distal end of the cannula during, this supporting and directing the cannula during insertion. 29. The needle may be arranged either within the cannula or the needle may be hollow and arranged outside the cannula.

In a yet further aspect an assembly is provided comprising a transcutaneous device unit and a process unit. The transcutaneous device unit is adapted for application towards a skin surface of a subject and comprises a housing, and an extendable transcutaneous device having a distal end portion adapted to be arranged through the skin of the subject at an inclined angle relative to the skin surface. The process unit is adapted to be releasably coupled to the housing, the process unit comprising a process assembly adapted for cooperation with the transcutaneous device, wherein the process unit in a situation of use in which the assembly has been applied towards the skin of a subject covers the cannula in its extended position, and wherein at least partial removal of the process unit from the transcutaneous device unit allows inspection of the introduction site of the transcutaneous device through the skin surface.

In an exemplary embodiment the transcutaneous device unit comprises a transcutaneous drug delivery device, and the process unit comprises a reservoir adapted to contain a fluid drug, and an expelling assembly adapted for cooperation with the reservoir to expel fluid drug out of the reservoir and through the skin of the subject via the transcutaneous drug delivery device when the two units are coupled to each other.

The transcutaneous drug delivery device may be in the form of e.g. a pointed hollow infusion needle or a combination of a relatively flexible per se blunt cannula with a penetrating insertion needle, the insertion needle being retractable after insertion of the blunt portion of the transcutaneous device. The cannula is advantageously soft and flexible relative to the insertion needle which may be a solid steel needle arranged inside the cannula or a hollow needle arrange outside the cannula. The length of the transcutaneous device may be chosen in accordance with the actual application, e.g. 4-20 mm. Indeed, the housing may comprise more than one transcutaneous drug delivery device.

To reduce the likelihood of transcutaneous device injuries, the distal end of the transcutaneous device may be moveable between the extended position in which the end projects relative to the mounting surface, and a retracted position in which the end is retracted relative to the mounting surface.

The term expelling assembly covers an aggregation of components or structures which in combination provides that a fluid can be expelled from the reservoir. The expelling assembly may e.g. be a mechanical pump (e.g. a membrane pump, a piston pump or a roller pump) in combination with electronically controlled actuation means, a mechanically driven pump (e.g. driven by a spring), a gas driven pump or a pump driven by an osmotic engine. The expelling assembly may also be in the form of an aggregation of components or structures which in combination provides that a fluid can be expelled from the reservoir when the expelling assembly is controlled or actuated by a controller external to the expelling assembly.

In a further exemplary embodiment the transcutaneous device unit comprises a transcutaneous sensor device and the process unit is adapted to transmit and/or process data acquired via the sensor. The penetrating sensor may allow a body parameter to be sensed in the subcutaneous space, e.g. by using a needle formed sensor as discussed in the introduction, or by transporting fluid from the subcutaneous space to detection assembly by means of a conduit, this principle being known as micro-dialysis. An example of a penetrating needle-sensor and a corresponding process unit is shown in U.S. Pat. No. 6,809,653 (hereby incorporated by reference) which discloses a characteristic monitor system including a data receiving device, a transcutaneous needle sensor for producing signal indicative of a characteristic of a subject (e.g. a blood glucose value), and a processor device. The processor device includes a housing, a sensor connector, a processor, and in the shown embodiment a transmitter. In the shown embodiment the processor coupled to the sensor processes the signals from the sensor for transmission to the remotely located data receiving device, however, the processed data could also be shown directly on a display provided on the processor device. The data receiving device may be a characteristic monitor, a data receiver that provides data to another device, a wireless programmer for a medical device (e.g. a remote control), a medication delivery device (such as an infusion pump), or the like.

The devices described above in accordance with individual aspects of the invention can be used both independently of each other and in combination with elements and features in accordance with other aspects of the invention.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject. Further, the term needle (when not otherwise specified) defines a piercing member adapted to penetrate the skin of a subject.

### BRIEF DESCRIPTION OF THE DRAWINGS

In the following the invention will be further described with reference to the drawings, wherein

FIGS. 1-3 shows in perspective views sequences of use for a first embodiment of a drug delivery device,

FIG. 4 shows in a non-assembled state a needle unit and a reservoir unit for a further embodiment of a drug delivery device,

FIG. 5 shows an exploded view of the needle unit of FIG. 4,

FIG. 6 shows a perspective view of the needle unit of FIG. 4 in a first state,

FIG. 7 shows a perspective view of the needle carrier of FIG. 5,

FIG. 8 shows a perspective view of the needle unit of FIG. 4 in a second state,

FIG. 9 shows a side view of the needle unit of FIG. 4,

FIG. 10 shows a further perspective view of the needle unit of FIG. 4,

FIG. 11 shows perspective view of the interior of the reservoir unit of FIG. 4,

FIG. 12 shows an exploded view of a further reservoir unit,

FIGS. 13A and 13B show in a schematic representation a transcutaneous device in the form of a cannula and insertion needle combination,

FIG. 14 shows a side view of a medical device mounted on a curved skin surface,

FIG. 15 shows medical device comprising a patch unit and an inserter unit,

FIG. 16 shows an exploded view of the device of FIG. 15,

FIG. 17 shows the device of FIG. 16 from below,

FIGS. 18A-18F show different states of use of the device of FIG. 15,

FIG. 19 shows an exploded view of a patch unit comprising an inserter assembly,

FIG. 20A shows in an exploded view details of the inserter assembly of FIG. 19,

FIG. 20B shows the details of FIG. 20A in an assembled state,

FIGS. 21A-21D show different states of use of the device of FIG. 19, and

FIG. 22 shows an alternative configuration for the device of FIG. 19.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

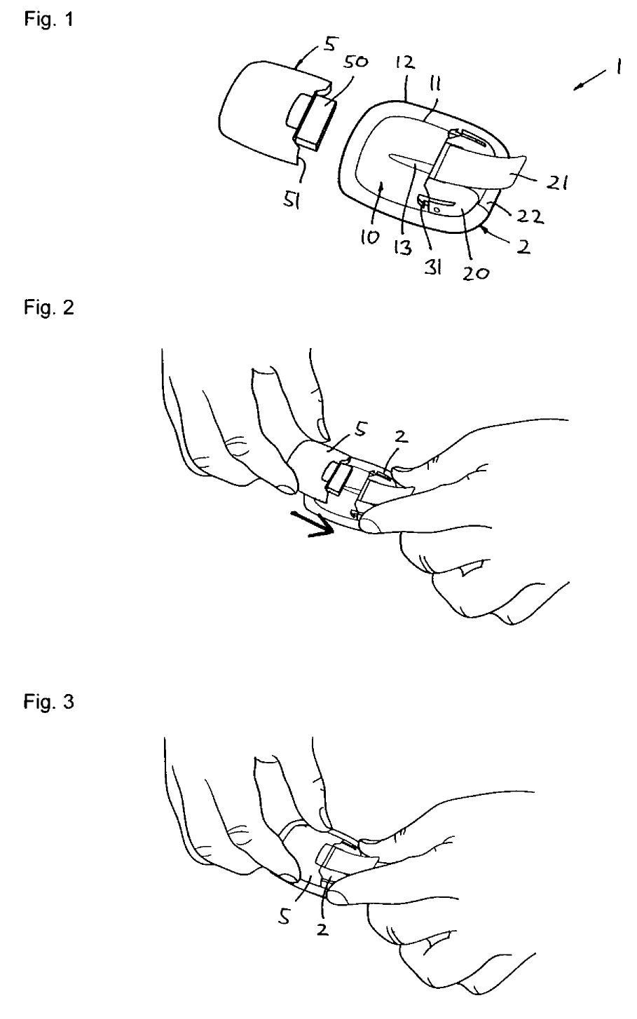

Firstly, with reference to FIGS. 1-3 an embodiment of a medical device for drug delivery will be described focusing primarily on the directly user-oriented features. The transcutaneous device unit 2 comprises a transcutaneous device in the form of a hollow infusion device, e.g. a needle or soft cannula, and will thus in the following be termed a needle unit, however, the needle may be replaced with any desirable transcutaneous device suitable for delivery of a fluid drug or for sensing a body parameter.

More specifically, FIG. 1 shows a perspective view of medical device in the form of a modular skin-mountable drug delivery device 1 comprising a patch-like needle unit 2 (which may also be denoted a patch unit) and a reservoir unit 5. When supplied to the user each of the units are preferably enclosed in its own sealed package (not shown). The embodiment shown in FIG. 1 comprises a patch unit provided with an insertable steel needle, however, the embodiment is exemplary of how to use a patch unit with an insertable transcutaneous device, e.g. needle, cannula or sensor. In case an actual embodiment requires the patch unit to be mounted on the skin and the transcutaneous device inserted before a reservoir or other unit can be attached, it follows that the method of use would be adopted correspondingly.

The needle unit comprises a flexible patch portion 10 with a lower adhesive mounting surface adapted for application to the skin of a user, and a housing portion 20 in which a hollow infusion needle (not shown) is arranged. The needle comprises a pointed distal end adapted to penetrate the skin of a user, and is adapted to be arranged in fluid communication with the reservoir unit. In the shown embodiment the pointed end of the needle is moveable between an initial position in which the pointed end is retracted relative to the mounting surface, and an extended position in which the pointed end projects relative to the mounting surface. Further, the needle is moveable between the extended position in which the pointed end projects relative to the mounting surface, and a retracted position in which the pointed end is retracted relative to the mounting surface. The needle unit further comprises user-gripable actuation means in the form of a first strip-member 21 for moving the pointed end of the needle between the initial and the second position when the actuation means is actuated, and user-gripable retraction in the form of a second strip-member 22 means for moving the pointed end of the needle between the extended and the retracted position when the retraction means is actuated. As can be seen, the second strip is initially covered by the first strip. The housing further comprises user-actuatable male coupling means 31 in the form of a pair of resiliently arranged hook members adapted to cooperate with corresponding female coupling means on the reservoir unit, this allowing the reservoir unit to be releasable secured to the needle unit in the situation of use. A flexible ridge formed support member 13 extends from the housing and is attached to the upper surface of the patch. In use a peripheral portion 12 of the patch extends from the assembled device as the reservoir unit covers only a portion 11 of the upper surface of the patch. The adhesive surface is supplied to the user with a peelable protective sheet.

The reservoir unit 5 comprises a pre-filled reservoir containing a liquid drug formulation (e.g. insulin) and an expelling assembly for expelling the drug from the reservoir through the needle in a situation of use. The reservoir unit has a generally flat lower surface adapted to be mounted onto the upper surface of the patch portion, and comprises a protruding portion 50 adapted to be received in a corresponding cavity of the housing portion 20 as well as female coupling means 51 adapted to engage the corresponding hook members 31 on the needle unit. The protruding portion provides the interface between the two units and comprises a pump outlet and contact means (not shown) allowing the pump to be started as the two units are assembled. The lower surface also comprises a window (not to be seen) allowing the user to visually control the contents of the reservoir before the two units are connected.

First step in the mounting procedure is to assemble the two units by simply sliding the reservoir unit into engagement with the needle unit (FIG. 2). When the hook members properly engage the reservoir unit a “click” sound is heard (FIG. 3) signalling to the user that the two units have been properly assembled. If desired, a visual or audible signal may also be generated. Thereafter the user removes the peelable sheet 14 to uncover the adhesive surface where after the device can be attached to a skin surface of the user, typically the abdomen. Infusion of drug is started by gripping and pulling away the actuation strip 21 as indicated by the arrow whereby the needle is inserted followed by automatic start of the infusion. The needle insertion mechanism may be supplied in a pre-stressed state and subsequently released by the actuation means or the needle insertion may be “energized” by the user. A “beep” signal confirms that the device is operating and drug is infused. The reservoir unit is preferably provided with signal means and detection means providing the user with an audible alarm signal in case of e.g. occlusion, pump failure or end of content.

After the device has been left in place for the recommended period of time for use of the needle unit (e.g. 48 hours)—or in case the reservoir runs empty or for other reasons—it is removed from the skin by gripping and pulling the retraction strip 22 which leads to retraction of the needle followed by automatic stop of drug infusion where after the strip which is attached to the adhesive patch is used to remove the device from the skin surface.

When the device has been removed the two units are disengaged by simultaneously depressing the two hook members 31 allowing the reservoir unit 5 to be pulled out of engagement with the needle unit 2 which can then be discarded. Thereafter the reservoir unit can be used again with fresh needle units until it has been emptied.

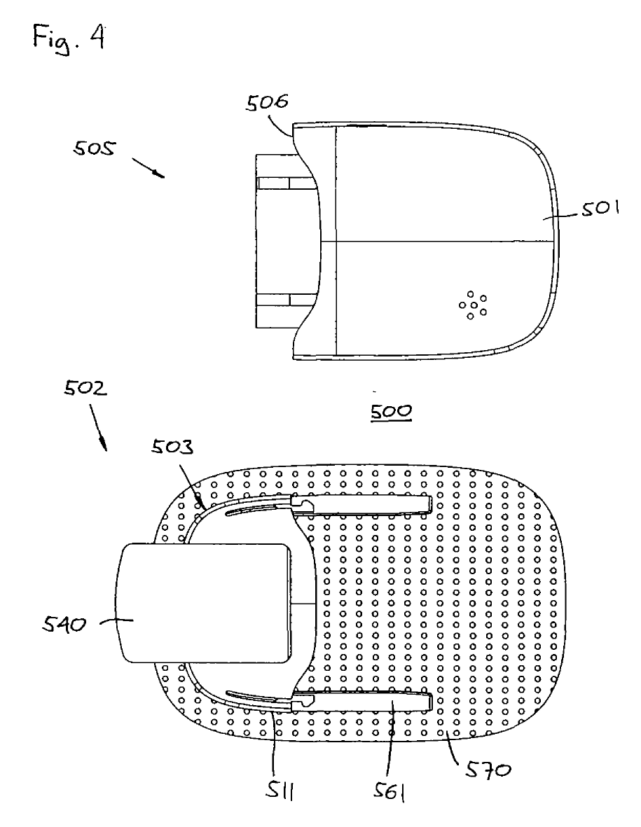

FIG. 4 shows a further embodiment of medical device 500 substantially corresponding to the embodiment of FIG. 1, the device comprising a transcutaneous device unit 502 and a process unit 505, More specifically, the transcutaneous device unit comprises a flexible patch portion (in the shown embodiment formed by a perforated sheet member 570) comprising an upper surface and a lower surface, the lower surface being adapted for application to the skin of a subject, a first housing 503 comprising a first coupling with two male coupling elements 511, and a transcutaneous device arranged in the housing (see below). Two supporting ridge members 561 extend from the first housing and are attached to the upper surface of the sheet member. The supports serve as attachment supports for the first housing, however, they may also serve to control the distance between the lower surface or the process unit and the patch. When the second unit is configured to accommodate at least partially the support members, e.g. in corresponding cut-out portions or grooves 504 (see FIG. 12), the supports may also serve to laterally stabilize the connection between the two units. The process unit comprises a second housing 501 with a lower surface and a second coupling arranged at a peripheral portion of the second housing, and a process assembly, e.g. a pump assembly as will be described below. In the shown embodiment the process unit has a generally flat rectangular shape with a cut-off end portion defining the interface with the transcutaneous device unit and also comprising the coupling in the form of two female coupling elements 506 arranged at each side of the end portion. Corresponding to FIGS. 1-3, the first and second couplings can be connected to each other with the upper surface of the patch facing towards the lower surface of the second housing. Due to the peripheral arrangement of the second coupling the flexible patch portion facing towards the lower surface of the second housing is free to move relative thereto, the degree of freedom being determined by the flexibility of the patch and supports if so provided and, of course, the surface to which the transcutaneous device unit is mounted.

In the shown embodiment the patch portion has the same general shape as the combined device albeit somewhat larger. In alternative embodiments the patch may comprise openings or cut-out portions. For example, an area between the two support legs may be cut out allowing the underlying skin to better breath.

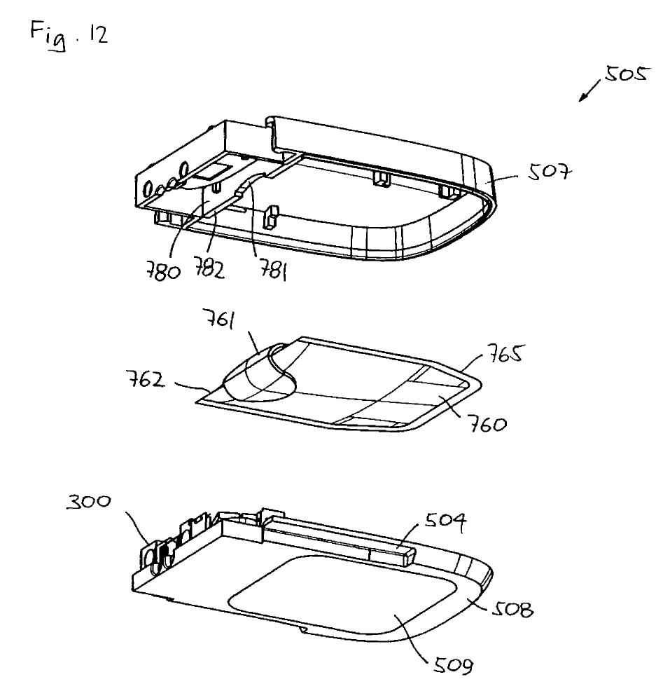

FIG. 14 shows a side view of the assembled device 500 mounted on a curving skin surface 590. As appears, the flexible patch portion with its support members is allowed to follow the curvature of the skin, this creating a ventilation space between the process unit and the patch portion.

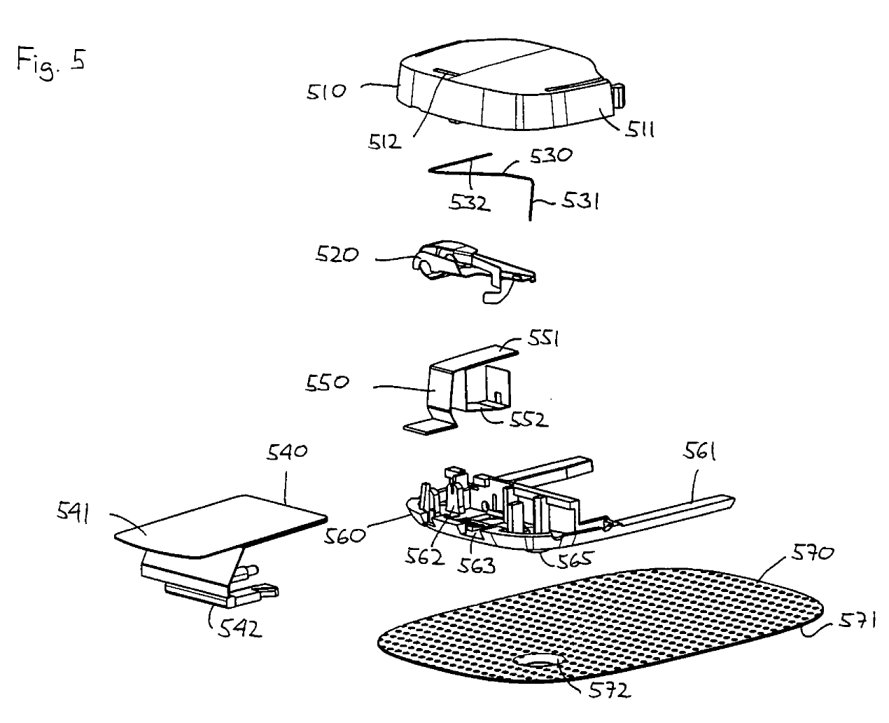

FIG. 5 shows an exploded perspective view of the needle unit comprising an upper housing portion 510, a needle carrier 520 and a thereto mounted infusion needle 530, an actuation member 540, a release member 550, a lower housing portion 560 and a sheet member 570. The actuation member comprises a user gripable portion 541 and a needle actuation portion 542, and the release member comprises a user gripable portion 551 and a needle retraction portion 552. In the assembled state as shown in FIG. 6, the upper and lower housing portions form a housing 503 in which the needle and the needle carrier is mounted, the actuation and release members being operatable connected to the needle carrier with the user gripable portions arranged outside the housing. The sheet member further comprises an opening 572 arranged in register with a lower protrusion 565 provided around the exit aperture for the transcutaneous device, just as the sheet is provided with a large number of small perforations to improve breathability through the sheet. The housing 503 is provided with user actuatable coupling means 511 allowing a reservoir unit to be attached to and released from the needle unit 505, the reservoir unit comprising corresponding mating coupling means 506 as well as a display 587. The display may indicate e.g. proper function of the unit, the amount of drug in the reservoir or different error conditions.

As seen is the user gripable portion 551 of the release member initially covered by a portion of the actuation member, this reducing the probability that the user erroneously uses the release member instead of the actuation member. Further, the actuation and release members (or portion thereof) may be colour coded to further assist the user to correctly use the device. For example, the actuation member may be green to indicate “start” whereas the release member may be red to indicate “stop”.

FIG. 7 shows in perspective the needle carrier 520 with the needle 530 and the needle actuation portion 542 of the actuation member 540. The needle actuation portion comprises two legs 543 allowing it to slide relative to the housing, the legs being arranged through respective openings 563 in the housing. The needle carrier is adapted to be connected to a hinge member 562 of the lower housing portion to thereby allow the needle carrier and thereby the needle to pivot corresponding to a pivoting axis defined by a hinge. In the shown embodiment is the needle carrier in the form a bent sheet metal member, the carrier comprising an upper arm 521 and a lower arm 522 connected to each other by a hinge portion 523 allowing the lower arm to pivot relative to the upper arm and corresponding to the pivoting axis. The lower arm forms a tray in which the hollow infusion needle 530 is mounted (e.g. by welding or adhesive), the needle having a distal pointed portion 531 adapted to penetrate the skin of the subject, the distal portion extending generally perpendicular to the mounting surface of the needle unit, and a proximal portion 532 arranged substantially corresponding to the pivoting axis and adapted to engage a fluid supply. Thus, when a portion of the upper arm is mounted in the housing, the lower arm can pivot between a first retracted position in which the distal portion of the needle is retracted within the housing, and a second extended position in which the distal portion projects relative to the mounting surface. In the shown embodiment the needle carrier provides the drive means for moving the lower arm between the two positions. This may as in the present embodiment be provided by the elastic properties of the sheet material per se corresponding to the hinge portion, or alternatively an additional spring may be provided between the two arms to thereby urge them apart. To lock the lower part in an energized, releasable first position, the upper arm is provided with a flexible release arm 526 comprising a catch 527 supporting and arresting the lower arm in its first downwardly biased position, as well as a release portion 528 engaging a ramp surface 544 of the needle actuation portion 542, the catch further comprising an inclined edge portion 529 adapted to engage the lower arm when the latter is moved from its extended to its retracted position as will be described in greater detail below.

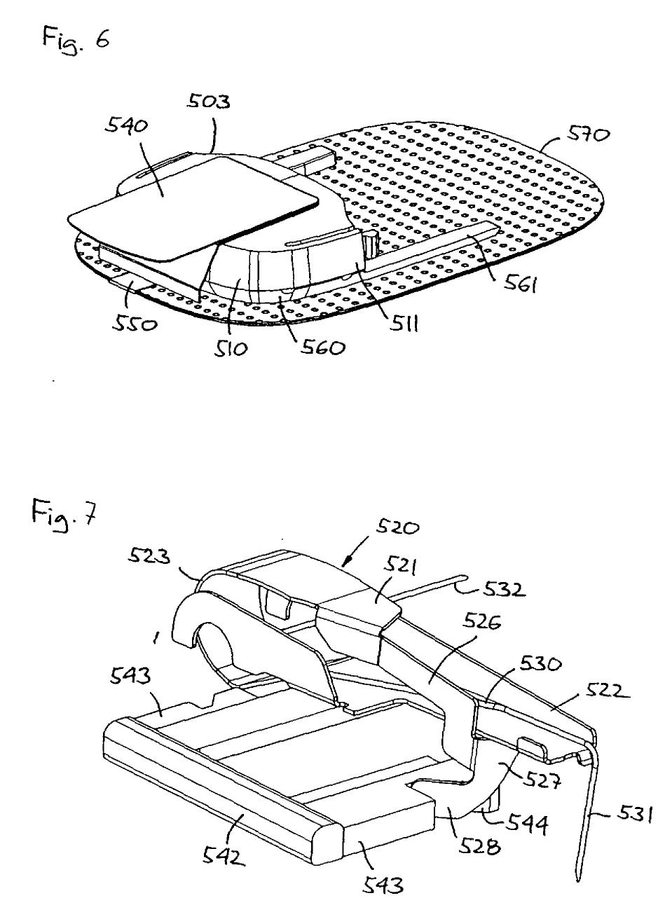

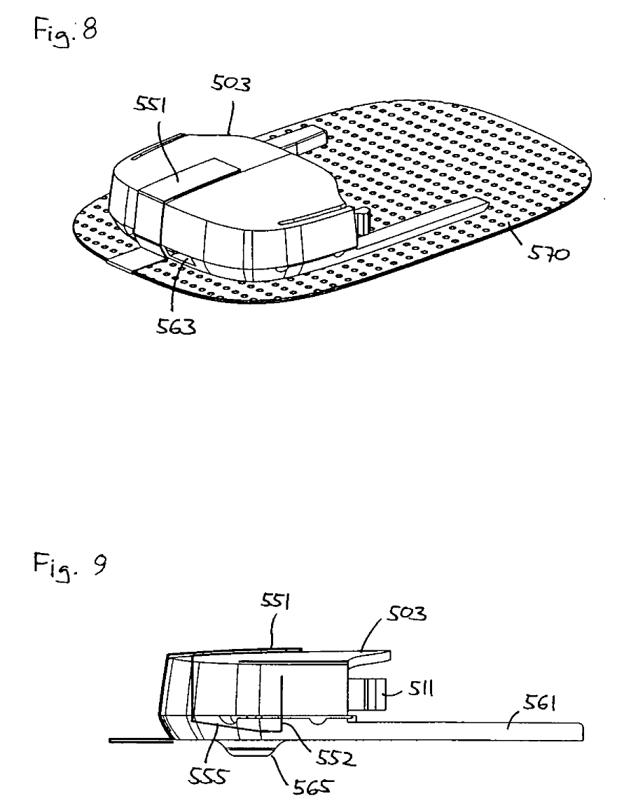

To actuate the needle the user grips the flexible strip forming the user gripable portion 541 (which preferably comprises adhesive portions to hold it in its shown folded initial position) and pulls the needle actuation portion 542 out of the housing, the actuation member 540 thereby fully disengaging the housing. More specifically, when the ramp surface 544 is moved it forces the latch 527 away from the lower arm to thereby release it, after which the release portion 528 disengages the ramp allowing the two legs to be pulled out of the housing. As seen in FIG. 8, when the actuation member is removed the user gripable portion 551 of the release member is exposed. As for the actuation member, the user gripable portion of the release member preferably comprises adhesive portions to hold it in its shown folded initial position.

In the shown embodiment the release member is in the form of a strip formed from a flexible material and having an inner and an outer end, the strip being threaded through an opening 512 in the housing, the strip thereby forming the user gripable portion 551 and the needle retraction portion 552, the inner end of the strip being attached to the housing and the outer end of the strip being attached to a peripheral portion of the sheet member 570 or, alternatively, a peripheral portion of the housing. In the projection shown in FIG. 9 the release member is shown in its initial position, the retraction portion forming a loop 555 arranged below the lower arm of the needle carrier, this position allowing the lower arm to be moved to its actuated position and thereby the needle to its extended position.

When the user decides to remove the needle unit from the skin, the user grips the user gripable portion 551, lifts it away from the housing and pulls it upwardly whereby the loop shortens thereby forcing the lower arm upwardly, this position corresponding to an intermediate release state. By this action the lower arm engages the inclined edge portion 529 of the catch 527 thereby forcing it outwardly until it snaps back under the lower arm corresponding to the position shown in FIG. 7. As the actuation member 540 has been removed from the needle unit, the needle carrier is irreversibly locked in its retracted position. When the user further pulls in the release member, the peripheral portion of the sheet member to which the release member is attached will be lifted off the skin, whereby the needle unit with its attached reservoir unit can be removed from the skin, this as described above.

Advantageously, the actuation and release members may be formed and arranged to communicate with the reservoir unit (not shown). For example, one of the legs of the actuation member may in its initial position protrude through the housing to thereby engage a corresponding contact on the reservoir unit, this indicating to the reservoir unit that the needle unit has been attached, whereas removal of the actuation member will indicate that the needle has been inserted and thus that drug infusion can be started. Correspondingly, actuation of the release member can be used to stop the pump.

In FIG. 10 the side of the needle unit 502 which connects to the reservoir unit is shown. In addition to the two ridge members 561 and the user actuatable coupling means 511 the needle unit comprises further structures which connects to and/or engages the reservoir unit to provide a functional interface with the reservoir unit. More specifically, the needle unit comprises a fluid inlet provided by the pointed proximal portion 532 of the needle projecting from the needle unit and adapted to engage a fluid outlet of the reservoir unit, an actuator 515 projecting from the needle unit and adapted to engage and actuate a fluid connector in the reservoir unit (see below), and first and second contact actuators 548, 558 adapted to engage corresponding contacts on the reservoir unit. The first contact actuator is provided by the distal end of one of the legs 543 of the needle actuator projecting through an opening in the housing, and the second contact actuator is provided by a hinged portion of the housing connected to the needle retraction portion 552 of the release member 550. When the needle unit is first connected to the reservoir unit both contact actuators will protrude from the housing and engage the corresponding contacts on the reservoir unit thereby indicating that that a needle unit has been connected. When the needle is actuated the first contact actuator will be withdrawn and thereby disengage the corresponding contact on the reservoir unit to start pump actuation. When the needle is retracted the second contact actuator will pivot and disengage the corresponding contact on the reservoir unit to stop pump actuation.

FIG. 11 shows the reservoir unit with an upper portion of the housing removed. The reservoir unit comprises a reservoir 760 and an expelling assembly comprising a pump assembly 300 and control and actuation means 580, 581 therefore. The pump assembly comprises an outlet 322 for connection to a transcutaneous access device (e.g. the needle 530) and an opening 323 allowing an internal fluid connector to be actuated, see below. The reservoir 560 is in the form of prefilled, flexible and collapsible pouch comprising a needle-penetratable septum adapted to be arranged in fluid communication with the pump assembly, see below. The shown pump assembly is a mechanically actuated membrane pump, however, the reservoir and expelling means may be of any suitable configuration.

The control and actuation means comprises a pump actuating member in the form of a coil actuator 581 arranged to actuate a piston of the membrane pump, a PCB or flex-print to which are connected a microprocessor 583 for controlling, among other, the pump actuation, contacts 588, 589 cooperating with the contact actuators on the needle unit, signal generating means 585 for generating an audible and/or tactile signal, a display (not shown) and an energy source 586. The contacts are preferably protected by membranes which may be formed by flexible portions of the housing.

In FIG. 12 an exploded view of the reservoir unit 505 of FIG. 4 is shown, the unit comprising an upper housing member 507, a lower housing member 508 with a transparent area 509 and grooves 504 to receive the ridge members 561 extending from the needle unit, a flexible reservoir 760 with a rounded edge portion 762 on which a septum member 761 is mounted, a pump assembly 300 with actuator and a circuit board (not shown) arranged above the reservoir and comprising electronic components for controlling actuation of the pump. The upper and lower housing members comprise reservoir mounting means in the form of opposed upper and lower ridge portions 780 (the lower not seen) adapted to engage and mount the reservoir in the housing. Each ridge portion comprises a central cut-out portion 781 adapted to engage the septum member on its opposed surfaces when the housing members are assemble thereby locking the reservoir in place within the housing. The degree of locking will be determined by the pressure exerted on the septum member, the elastic properties of the septum member and the friction between the ridge and the septum member. On each side of the cut-out portion the ridge portions comprise a straight portion 782 which may aid in mounting the reservoir in the housing. The straight portions may engage the initially prefilled reservoir to help lock it in place, however, as the reservoir is emptied and flattens this grip may lessen. In contrast, the engagement with the septum is adapted to properly hold the reservoir in place as the reservoir is emptied. The straight portions may also be adapted to pinch and fully flatten the reservoir thus serving as an additional mounting means. Additional mounting means (not shown) may engage and grip the reservoir at other locations, e.g. along the welded edges 765.

In the above described embodiments, the transcutaneous device has been in the form of a unitary needle device (e.g. an infusion needle as shown or a needle sensor (not shown)), however, the transcutaneous device may also be in the form of a cannula or a sensor in combination with an insertion needle which is withdrawn after insertion thereof. For example, the first needle portion may be in the form of a (relatively soft) infusion cannula (e.g. a Teflon®(cannula) and a there through arranged removable insertion needle. This type of cannula needle arrangement is well known from so-called infusion sets, such infusion sets typically being used to provide an infusion site in combination with (durable) infusion pumps.

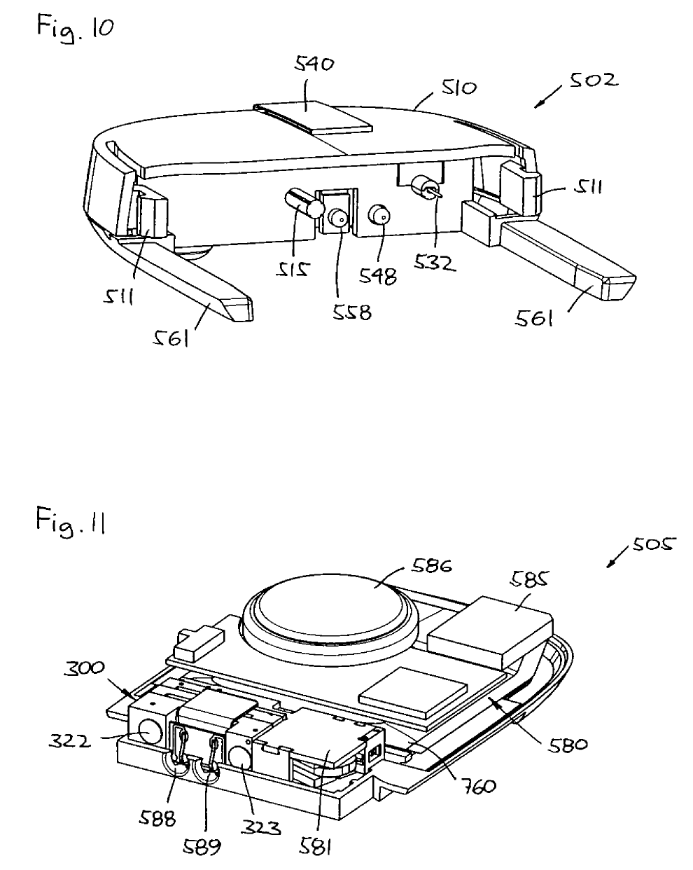

Thus, FIGS. 13A and 13B show in a schematic representation how a cannula and insertion needle combination can be arranged within a housing 601 of in a given medical device 600 (partly shown), e.g. an infusion device or an infusion set. More specifically, the medical device comprises a transcutaneous assembly 650 comprising a combination of a relatively soft cannula 651 (which e.g. may be of the soft “Teflon®” type) carried by a lower member 653 and a pointed insertion needle 661 (e.g. made from medical grade stainless steel) slidably arranged within the cannula and carried by an upper member 663, both members being mounted to allow axial displacement of the cannula respectively the insertion needle. The cannula comprises a proximal inlet (not shown) allowing it to be or to be arranged in fluid communication with a fluid source. The medical device further comprises a base plate 620 with an opening 621 for the cannula as well as a release member 622. The lower member comprises an elastomeric seal 652 through which the insertion needle is arranged. The cannula and the insertion needle may be straight or curved dependent upon how the two members are mounted in the device, e.g. arcuate corresponding to a pivoting axis or straight corresponding to linear movement as illustrated. The upper member comprises a coupling member 667 locking the members together in an initial position with distal end of the insertion needle extending from the distal opening of the cannula as shown in FIG. 13A, and the base plate comprises coupling member 657 for locking the lower member in an extended position with distal end of the cannula extending through the opening in the base plate (see FIG. 13B). Between the housing of the device and the upper member a first spring 668 is arranged biasing the upper member upwards. Correspondingly, the device also comprises a second spring 658 biasing the lower member upwardly. The medical device further comprises a gripping tab 676 and a pulling member 677 corresponding to the embodiment shown in FIG. 1.

In a situation of use the assembly is moved downwardly, either manually or by a releasable insertion aid, e.g. a spring loaded member acting through an opening in the housing (not shown) whereby the cannula with the projecting insertion needle is inserted through the skin of a subject. In this position the lower member engages the coupling member 657 to thereby lock the cannula in its extended position, just as the coupling member 667 is released by the release member 622 thereby allowing the upper member to return to its initial position by means of the first spring.

When the user intends to remove the delivery device from the skin surface, the user grips the gripping portion of the tab and pulls it in a first direction substantially in parallel with the skin surface, by which action the flexible strip 677 releases the coupling member 657 from the lower member whereby the lower member and thereby the cannula is retracted by means of the second spring. When the cannula has been withdrawn from the skin, the user uses the now unfolded tab to pull off the entire delivery device from the skin surface, for example by pulling the tab in a direction away from the skin surface.

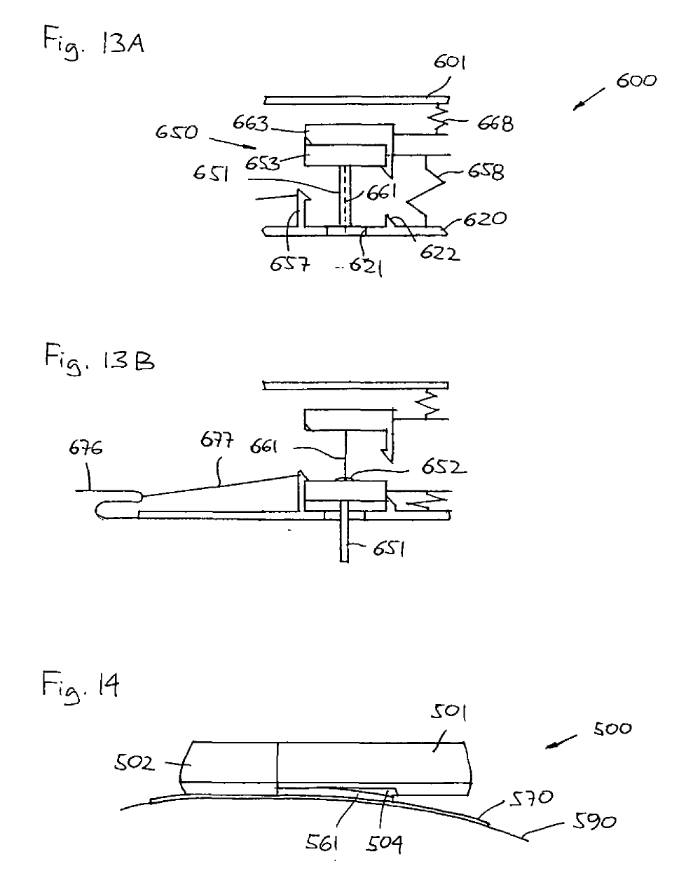

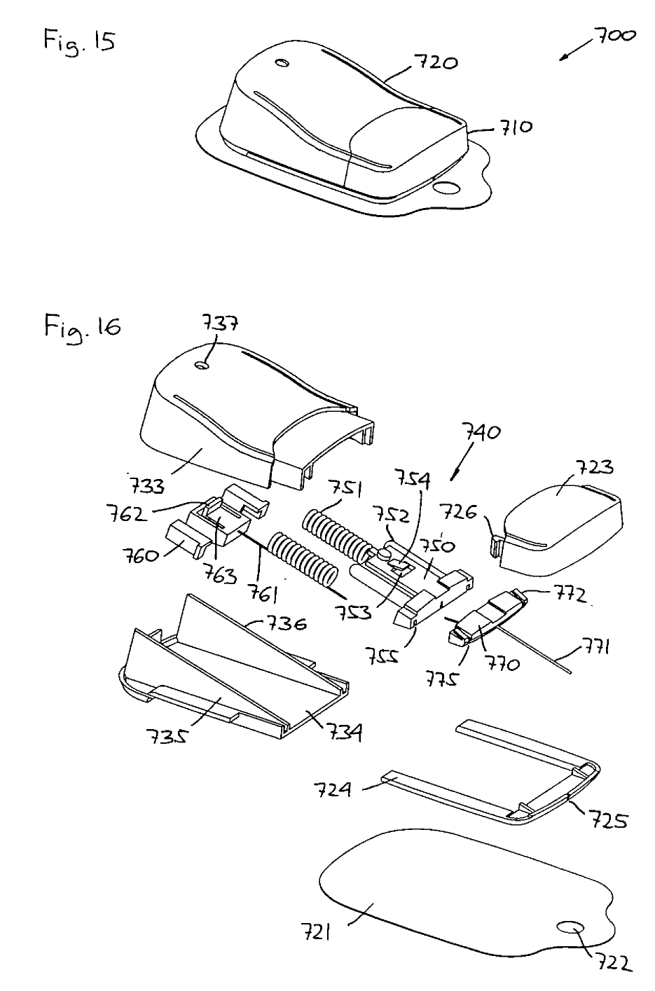

With reference to FIGS. 15-18 a medical device 700 will be described comprising a cannula and an insertion needle (in the following also “needle” for short). The cannula may be in the form of what is traditionally referred to as a “soft catheter” or a “Teflon® catheter”. The device comprises two portions, a patch unit 710 comprising a housing mounted on a patch of flexible sheet material, and an inserter unit 720 removeably coupled to the patch housing. The inserter housing initially comprises the entire insertion mechanism including the cannula. When actuated the cannula becomes attached to the patch housing where after the inserter housing with the remaining inserter mechanism can be detached and discarded.

More specifically, the patch unit comprises a flexible sheet 721 with a lower adhesive surface and an opening 722 for the cannula, a patch housing with top 723 and base 724 portions, the base portion being attached to the upper surface of the sheet. The patch housing comprises an opening 725 for the cannula arranged just above the opening in the sheet, as well as a coupling in the form of two flexible arms 726 allowing the inserter to be attached.

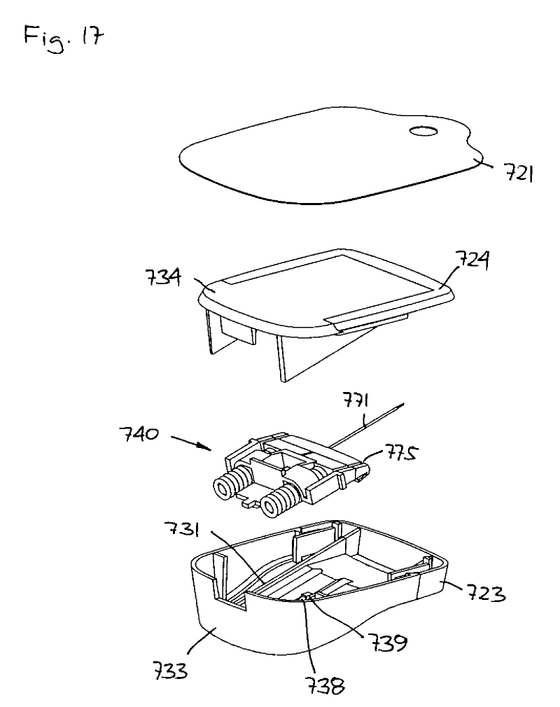

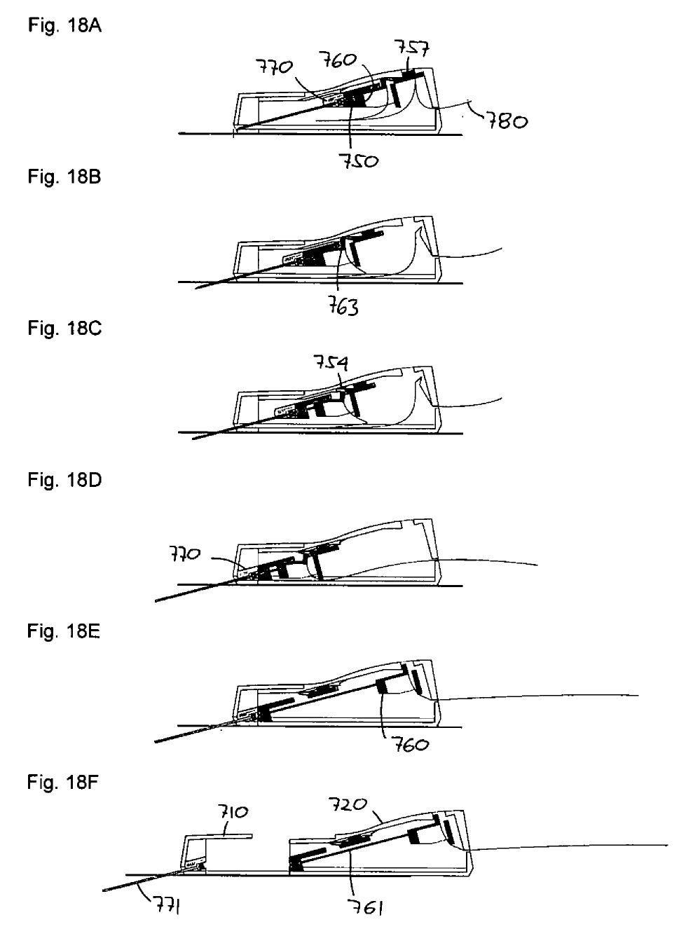

The inserter unit comprises an inserter housing with top 733 and base 734 portions, the base portion comprising two walls 735 with upper inclined edges serving as a ramp 736 for an inserter assembly 740. The inserter assembly comprises an inserter 750, a needle holder 760 comprising a needle 761 protruding there from, a cannula holder 770 with a cannula 771 protruding there from, the cannula comprising a proximal needle penetratable septum, two springs 751 mounted on respective spring guides 752 on the inserter, and a release and retraction strip 780 (see FIG. 18A). The strip comprises a proximal end projecting from the housing and a distal end attached to the needle holder, the strip forming a loop portion attached to the inserter. The inserter and the cannula holder are each provided with pairs of grooves 755, 775 allowing the inserter and the cannula to slide on the ramp. The inserter comprises an opening 753, an inclined ramp member 754 and a locking projection 757 adapted to engage a corresponding opening 737 in the housing. The needle holder comprises a flexible release arm 763 with an upwardly protruding catch 762, and the cannula holder comprises a pair of coupling elements 772 for engagement with the patch housing. In an initial assembled state (see FIG. 17) the cannula holder is arranged in front of the inserter and the needle holder is arranged below the inserter with the needle positioned through the septum and within the cannula and projecting there from, and with the catch 762 protruding through the opening 753. As an example, the cannula may be a soft catheter with an OD of 0.7 mm and an ID of 0.4 mm and the needle may have an OD of 0.4 mm (G27). The inner surface of the inserter housing comprises a ramp 738 and a hold 739 adapted to engage the inserter assembly as described below. In a fully assembled initial state the inserter assembly is locked in place by the locking projections 757 engaging the opening 737 in the inserter housing, the springs being arranged in a compressed state between the inserter and the inserter housing. Upper guides 731 in the inserter housing secures that the inserter assembly can move only along the inclined ramp.

Next, with reference to FIGS. 18A-18F operation of a medical device of the above-outlined construction for insertion of a soft catheter will be described. The user first removes a protective sheet covering the adhesive surface of the patch and arranges the patch on a suitable skin portion of a subject, e.g. the abdomen. In the start position (see FIG. 18A) the soft catheter holder with a soft catheter is placed in front of the inserter. The needle holder is connected to the inserter, which is loaded with springs (see FIG. 15), all integrated in the inserter housing. The inserter needle is arranged inside the soft catheter with its pointed needle tip e.g. 2 mm in front of the soft catheter. Next the user pulls the strip which releases the inserter from the housing, this allowing the inserter assembly with soft catheter and needle holder to start move forwards pushed by the springs. As appears, by this action the strip is released from the needle holder. By the initial travel of the inserter assembly the inserter needle with soft catheter penetrates dermis 2-4 mm. During this movement the catch of the release arm on the needle-holder engages the ramp placed on the inserter-housing (see FIG. 18B). The ramp depresses the release arm in relation to its engagement with the inserter, and finally arrests the release arm as it engages the hold at the end of the ramp, this temporary halting movement of the needle holder. After needle movement has come to a halt, the inserter and the soft catheter holder continue forward movement driven by the springs, thereby moving the soft catheter ahead of the needle an into sub-cutis. The needle holder is stopped until the soft catheter tip is e.g. 1-5 mm in front of needle tip. During this movement the release arm on the needle holder is stopped by the hold in the housing, however, at the same time the flexible arm is engaged by the ramp member on the inserter. This ramp depresses the release arm until it is lifted free of the hold where after it again engages the inserter (see FIG. 18C). After the needle holder has re-engaged the inserter, the needle now follows the soft catheters movement through sub-cutis to a final position, and the needle can therefore act as guide for the soft catheter, with the tip of the needle e.g. 1-5 mm behind the tip of the soft catheter. When the cannula reaches its final fully extended position the soft catheter holder is positioned in the patch-housing where it is locked in place (see FIG. 18D). As appears, the above-described actions all take place automatically driven by the springs and in a very short time, this providing minimum discomfort to the subject.

At this point the soft catheter has been placed at the desired place and what remains is for the user to withdraw the needle and remove the remaining inserter assembly and housing. In the shown embodiment the inserter is locked in place in its foremost position. The needle holder is released from the inserter and the needle is retracted by the user pulling the strip attached to the needle holder until the needle has been locked in its fully retracted position with the distal pointed end arranged within the inserter housing (see FIG. 18E). In the shown embodiment the inserter serves to surround and protect the pointed end of the needle. Finally the user detaches the inserter housing from the patch unit which can then be disposed off (see FIG. 18F). The cannula is now ready to be connected to a fluid source, e.g. a reservoir unit as shown in FIG. 1 and of the same principal configuration as described with reference to FIGS. 11 and 12. Indeed, the interface of the pump assembly 300 will have to be modified in order to connect to the proximal septum of the soft catheter or cannula instead of a pointed needle end, i.e. the pump assembly will be provided with a pointed hollow needle establishing a fluid communication between the pump assembly and the inserted cannula.

With reference to FIGS. 15-18 an embodiment comprising a separate cannula inserter has been described, however, a corresponding mechanism may also be incorporated in a unitary patch unit. Such a design would indeed result in a larger patch housing, however, the user would not have to detach and discard the inserter. For such a design the needle may be hollow and comprise a proximal end, with the distal end of the needle being in sealed fluid communication with the interior of the cannula when the needle has been arranged in its retracted position. By this arrangement a fluid communication can be provided between the proximal end of the needle and the cannula, this allowing the fluid communication to be established between the patch unit and the reservoir unit corresponding to the connection between the units in the FIGS. 5-12 embodiment. In this case a delivery device would supply drug to the cannula via the hollow needle.

With reference to FIGS. 19-22 a further integrated concept will be disclosed. The concept consists of an introducer needle surrounding a cannula, e.g. a soft catheter. The 1-2 mm cutis or derma is penetrated by the needle and only the soft catheter is inserted into sub-cutis. Once the soft catheter is fully inserted, the needle is retracted. Since the needle is placed on the outside of the soft catheter, the soft catheter can be made in a smaller diameter compared to a concept in which the needle is arranged inside the needle and trauma in subcutis is thereby minimized, however, the larger diameter needle may cause larger trauma in the derma just as the cannula may be more susceptible to kinking and there may be less control when positioning the soft catheter in the subcutis. Also clotting during use may be more likely. These issues have to be considered when deciding on a specific concept and the specific design parameters for such a concept.

Turning to an exemplary embodiment, the medical device is in the form of a unitary patch unit 800 comprising a housing mounted on a patch of flexible sheet material, the inserter housing comprising the entire insertion mechanism including the cannula.

More specifically, the patch unit comprises a flexible sheet 821 with a lower adhesive surface and an opening 822 for the cannula (in this embodiment a flexible soft catheter), a patch housing with top 823 and base 824 portions (823′ indicates a top portion shown upside down), with the base portion being attached to the upper surface of the sheet, wherein the top portion comprises a 45 degrees guide 825 for the cannula holder (see below). The patch housing comprises an opening for the cannula and needle arranged just above the opening in the sheet, as well as a coupling in the form of two flexible arms 826 allowing a delivery device to be attached. The base portion comprises two walls 835 with upper inclined edges serving as a ramp 836 for an inserter assembly 840. The inserter assembly comprises an inserter 850 with an attached needle 861 and a cannula holder 870 attached to a cannula 871 and adapted for moving the cannula relative to the inserter and thereby the needle (see FIGS. 20A and 20B). The inserter is provided with pairs of grooves allowing the inserter to slide on the ramp. The insertion mechanism further comprises a user-releasable spring (not shown) for moving the inserter and a strip (not shown) for moving the cannula holder relative to the inserter. As an example, the soft catheter may have an OD of 0.4 mm and an ID of 0.1 mm and the needle may have an OD of 0.7 mm and an ID 0.4 mm (G22).

To save space in the patch housing, the soft catheter introducing mechanism is placed perpendicular in respect of the direction of introduction. The soft catheter 871 is placed in a groove 855 in the inserter that guides the soft catheter, the groove having a 90 degrees bend to change the direction of the soft catheter during the introduction. As appears from FIGS. 20A and 20B when the catheter holder 870 is moved across the inserter the soft catheter is extended in a perpendicular direction.

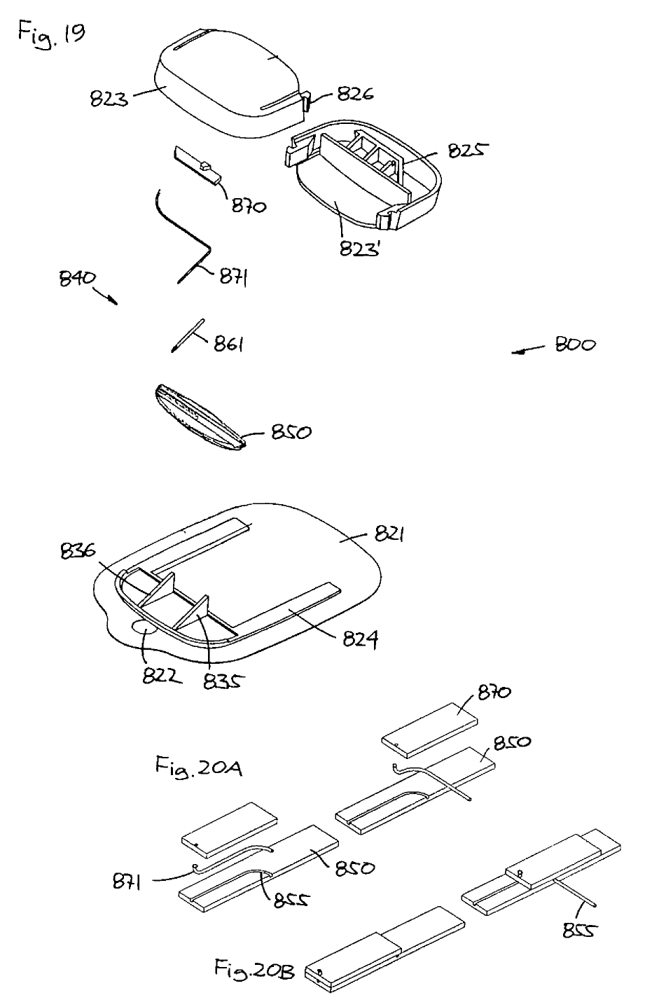

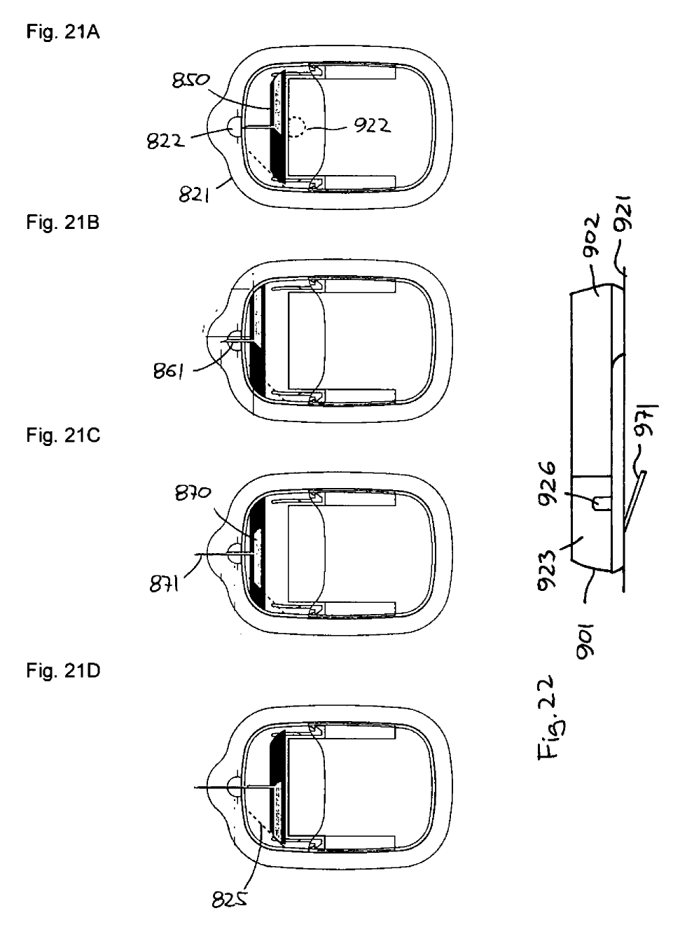

Next, with reference to FIGS. 21A-21D operation of a medical device of the above-outlined construction for insertion of a soft catheter will be described. The user first removes a protective sheet covering the adhesive surface of the patch and arranges the patch on a suitable skin portion of a subject, e.g. the abdomen. In the start position (see FIG. 21A) the inserter is arranged in its retracted position and the cannula holder is arranged in its initial position. When the inserter is released (e.g. by pulling a strip to release a spring) the introducer needle with the soft catheter inside penetrates dermis e.g. 2-4 mm (see FIG. 21B). By continuous pulling the strip the user starts the introducing of the soft catheter into sub-cutis by pulling the soft catheter holder across the inserter until the soft catheter is fully introduced (see FIG. 21C). After the soft catheter is fully introduced the user continues the pulling of the strip and pulls the soft catheter holder further across the inserter, however, as the soft catheter holder has reached the 45 degrees ramp, the inserter is forced backwards with the same speed as the soft catheter is moved forward, the result is that the soft catheter stays in its final position and the introducer needle on the inserter is removed and disappears into the patch housing (see FIG. 21D). If the cannula is only to be inserted for a relatively short period of time, or a relatively flexible needle is used, the device may be left in the state as shown in FIG. 21C without withdrawing the needle.

As the proximal end of the soft catheter is stationary, it may be provided with a pointed hollow needle which would allow a reservoir unit basically as shown in FIGS. 11 and 12 to be connected thereto.

FIG. 22 shows an alternative configuration for the device disclosed in FIG. 19. As the former embodiment the patch unit 901 comprises a flexible sheet 921 with a lower adhesive surface and an opening for the cannula, a patch housing 923 attached to the upper surface of the sheet and comprising an opening for the cannula, as well as a coupling in the form of two flexible arms 926 allowing a delivery device unit 902 to be attached. However, in contrast to the former embodiment the orientation of the angled cannula 971 has been reversed so that it points essentially in the opposite direction, i.e. towards the attached delivery device unit instead of away from the patch unit. Correspondingly, the opening in the flexible sheet is not peripherally but more centrally located (as indicated with dotted lines 922 in FIG. 21A). As appears, this arrangement allows the point of insertion of the cannula through the skin to be hidden and thus protected by the attached delivery device during normal operation of the assembled device, yet allows the cannula insertion site to be inspected by simply detaching and reattaching the delivery device unit. Further, as the modified inserter is moved towards the delivery device unit this movement may be used to connect the fluid inlet of the cannula with the fluid outlet from the delivery device unit, e.g. by means of a pointed needle connector and a needle penetratable septum arranged on either of the units. As appears, such a reversed arrangement may also be provided for a cannula inserter of the type disclosed with reference to FIGS. 15-18.

Indeed, the concept of a medical device comprising an angled insertable cannula which in its inserted position is covered by a detachable portion of the device can be used in combination with any type of cannula-needle arrangement, not only the embodiments disclosed above. The assembly may also be provided as a unitary device in which an opening may be formed allowing the insertion site to be inspected during use.

Although it is believed that the above-disclosed medical devices can be manufactured in a cost-effective manner, frequent changes of cannula or needle devices, e.g. infusion sets, is one of the cost drivers and poor convenience factors in CSII (continuous subcutaneous insulin infusion) treatment. It is today generally not recommended to wear an infusion set for more than 2 days before changing it, but in practice pump users wear them for a longer time—on average 3.3 days. One of the limiting factors in wear time is that the risk of bacterial growth at the infusion site increases with longer wear times. The preservatives in insulin are anti-bacterial, but since they don't get in touch with the outside of the infusion needle they have no effect on this bacterial growth.

With a porous infusion needle or cannula having a pore size between the molecular size of the preservatives (typically small molecules like meta-cresol and phenol) and the molecular size of insulin (rather large molecules), some of the preservatives will move to the outside of needle where they can reduce bacterial growth and potentially increase the safe wear time of the infusion needle. For a polymeric cannula the entire tube or portions thereof thus can advantageously be made from a polymeric material allowing the preservatives to diffuse from the cannula and into the subcutis. A cannula may also be made from a fibrous material as used in micro tubes for dialysis. For a steel needle laser drilling of micro side openings would allow preservatives together with insulin to diffuse out in the subcutis along the needle (unless the side openings are made so small that they would be an effective barrier to the insulin molecules). The porous portion of the needle may be uniformly porous or it may be adapted to cause weeping at a non-uniform flow rate along the length of the porous portion. A porous portion may e.g. be located at the portion of the needle or cannula intended to cross the skin barrier.

US 2004-0220536, which is hereby incorporated by reference, discloses a surgical needle with a porous distal portion from which a liquid injectate will weep or ooze multidirectionally under injection pressure while the porous distal portion of the needle is inserted into a body surface. More specifically, it is disclosed how a needle or cannula can be provided with pores from which a liquid will ooze. For example, the porous portion of the needle can be fabricated from any of a number of different “open cell” porous materials (i.e., materials in which the pores are interconnecting). For example, a distal portion can be fabricated from a porous sintered metal, such as forms a non-woven matrix of metal fibers selected from such metals as stainless steel, tantalum, elgiloy, nitinol, and the like, and suitable combinations of any two or more thereof. Generally, the metal fibers will have a diameter in the range from about 1.0 micrometer to about 25 micrometer. A non-woven matrix of metal fibers having these desired properties that can be used in manufacture of the porous distal portion of the invention needle is available from the Bekaeart Corporation (Marietta, Ga.), and is sold under the trademark, BEKIPOR® filter medium. A porous portion of the needle can also be fabricated from such porous materials as a porous polymer, such as a porous polyimide, polyethylene, polypropylene, polytetrafluroethylene, and the like. Such porous polymers are disclosed, for example, in U.S. Pat. No. 5,913,856, which is hereby incorporated by reference in its entirety. Alternatively, a porous ceramic can be used, such as is known in the art for use in ceramic filters and separation membranes, or a porous metal (also known as an expanded metal) or carbon, such as is known in the art for use in filters or bone grafts. For example, Mott Corporation (Farmington, Conn.) manufactures porous metals for use in various types of filters. If the porous filter medium is flexible, a porous portion of a needle can be fabricated by wrapping the filter medium, which is available commercially as a flat sheet, one or more times around an axis while creating a hollow central core. The porous portion of the needle can then be fused in fluid-tight fashion (e.g. welded) to a non-porous hollow needle shaft using methods known in the art. To create a porous portion of the needle having decreasing impedance to fluid flow, a porous filter medium or metal mesh having an appropriate porosity gradient can be employed in fabrication of the porous portion. Alternatively, a porous portion can be created from a non-porous material (e.g., a metal) using a cutting laser and techniques known in the art to punch pores into the needle segment (i.e. by a process of laser etching). For example, the nonporous hollow shaft, porous portion, and point of a needle can be fabricated of metal in a single piece, for example, from a conventional hypotube. In this scenario, a metal-cutting laser is used to create a segment of the needle that has appropriate porosity, for example, a porosity gradient within a portion of the needle to equalize fluid impedance along the length of the porous portion of the needle.

The direct advantage of the above principle is a reduced bacterial growth at the infusion site compared with standard infusion needles. This increases user convenience, since an infusion set can be worn longer before it needs to be replaced—a replacement that can be painful especially for soft infusion needles where a large diameter steel needle is used to guide the soft infusion needle into the skin. Since infusion sets are typically rather expensive, increased wear time will furthermore be cost-attractive to pump users.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A medical device (700, 800), comprising:
a housing adapted for application towards the skin of a subject,
a cannula (771, 871) having a distal end portion adapted to be arranged through the skin of the subject and having a distal opening, and
a needle (761, 861) arranged coaxially with and being axially moveable relative to the cannula, the needle comprising a distal end adapted to penetrate the skin of the subject,
wherein the medical device is transformable between:
a first state in which the cannula and the needle are retracted within the housing,
a second state in which the cannula and the needle are extended relative to the lower surface with the distal end of the needle projecting relative to the distal opening of the cannula thereby allowing the cannula to be introduced through the skin of the subject,
a third state in which the distal end of the needle is positioned short of the distal end portion of the cannula, the cannula not being fully extended relative to the housing, and
a fourth state in which the cannula is fully extended relative to the housing.

2. A medical device as in claim 1, wherein the needle (761) is arranged within the cannula (771).

3. A medical device as in claim 2, wherein in the fourth state the distal end of the needle is positioned within the cannula short of the distal opening.

4. A medical device as in any of claim 1, the medical device being transformable to a fifth state in which the needle is retracted from the portion of the cannula extending from the housing.

5. A medical device as in claim 3, comprising an inserter assembly (740) for moving the cannula and the insertion needle between the different states, the inserter assembly comprising:
the cannula (771),
an inserter (750) for moving the cannula,
the needle (761), and
a needle holder (760) attached to the needle,
wherein the inserter assembly has:
an initial state in which the needle holder is locked to the inserter in an initial position with the distal end of the needle projecting from the distal opening of the cannula, and
an intermediate state in which the needle holder is locked to the inserter in an intermediate position with the distal end of the needle positioned within the cannula short of the distal opening.

6. A medical device as in claim 5, wherein:
the inserter assembly is transformable from the first to the second state with the inserter assembly in its initial state,
the inserter assembly is transformable from the second to the third state when the inserter is transformed from its initial to its intermediate state, and
the inserter assembly is transformable from the third to the fourth state with the inserter assembly in its intermediate state.

7. A medical device as in claim 5, wherein the inserter assembly has a retracted state in which the needle is retracted from the portion of the cannula extending from the housing.

8. A medical device as in claim 6, wherein the inserter assembly is transformable from the fourth to the fifth state when the inserter is transformed from its intermediate to its retracted state.

9. A medical device as in claim 5, wherein the cannula in the fully extended position is adapted to be locked in place relative to the housing.

10. A medical device as in claim 5, wherein the inserter assembly further comprises a cannula holder (770) attached to the cannula, the cannula holder being moved by the inserter from a retracted to a fully extended position, the cannula holder and the housing being adapted for locking the cannula in its extended position.

11. A medical device as in claim 5, comprising a user-releaseable actuator (751) for actuating the inserter assembly from the first to the fourth state.

12. A medical device as in claim 11, wherein the actuator comprises a spring urging the inserter from an initial position to an extended position corresponding to the first respectively the fourth state.

13. A medical device as in claim 11, comprising a retractor (780) for retracting the needle from its fully extended to a retracted position.

14. A medical device as in claim 10, comprising first (723) and second (733) housing portions coupled to each other, wherein:
the cannula in its extended position is locked to the first housing portion,
the needle is retracted into the second housing portion, and
the second housing portion can be detached from the first housing portion, the needle being arranged there within.

15. A medical device as in claim 14, further comprising a flexible sheet member (721) with a lower surface adapted to be arranged on a skin surface of a subject, and an upper surface to which the first housing portion is arranged.

16. An assembly comprising a medical device as in claim 15 and a delivery device (5) adapted to be coupled to the first housing portion, the delivery device comprising a reservoir (760) adapted to contain a fluid drug, and an expelling assembly (300) adapted for cooperation with the reservoir to expel fluid drug out of the reservoir and through the skin of the subject via the cannula when the delivery device has been coupled to the first housing portion after the cannula has been inserted and the second housing portion removed.

17. A medical device as in claim 1, wherein the needle is hollow and comprises a proximal end, the distal end of the needle being in sealed fluid communication with the interior of the cannula with the needle in its retracted position, whereby a fluid communication is provided between the proximal end of the needle and the cannula.

18. An assembly comprising a medical device as in claim 17 and a delivery device adapted to be coupled to the first housing portion, the delivery device comprising a reservoir adapted to contain a fluid drug, and an expelling assembly adapted for cooperation with the reservoir to expel fluid drug out of the reservoir and through the skin of the subject via the hollow needle and the cannula when the delivery device has been coupled to the first housing portion after the cannula has been inserted and the needle retracted.

19. A medical device (800) as in claim 1, wherein the needle (861) is hollow and arranged outside the cannula (871).

20. A medical device as in claim 19, wherein the needle is fully extended corresponding to the third state.

21. A medical device as in claim 19, comprising an inserter assembly (840) for moving the cannula and the insertion needle between the different states, the inserter assembly comprising:
the cannula (871),
the needle (861),
an inserter (850) attached to the needle,
a cannula holder (870) attached to the cannula and adapted for moving the cannula relative to the inserter and thereby the needle,
wherein the inserter assembly has:
an initial state in which the cannula is positioned within the needle and with the distal end of the needle projecting relative to the distal opening of the cannula,
an intermediate state in which the cannula holder has been moved to extend the cannula from the needle, and
wherein the inserter assembly has a retracted and an extended position.

22. A medical device as in claim 21, wherein:
the inserter assembly is transformable from the first to the second state when the inserter assembly is moved from the retracted to the extended position with the inserter assembly in its initial state,
the inserter assembly is transformable from the second to the third state when the inserter is transformed from its initial to its intermediate state, and
the inserter assembly is transformable from the third to the fourth state with the inserter assembly in its intermediate state.

23. A medical device as in claim 21, wherein the inserter assembly has an extended state in which the cannula holder has been moved to further extend the cannula from the needle.

24. A medical device as in claim 23, wherein:
the inserter assembly is transformable from the first to the second state when the inserter assembly is moved from the retracted to the extended position with the inserter assembly in its initial state,
the inserter assembly is transformable from the second to the third state when the inserter is transformed from its initial to its intermediate state,
the inserter assembly is transformable from the third to the fourth state with the inserter assembly in its intermediate state, and
the inserter assembly is transformable from the fourth to the fifth state when the inserter is transformed from its intermediate to its extended state and when the inserter assembly is moved from the extended to the retracted position.

25. A medical device as in any of claim 21, further comprising a flexible sheet member (821) with a lower surface adapted to be arranged on a skin surface of a subject, and an upper surface to which the housing is arranged.

26. An assembly comprising a medical device as in claim 25 and a delivery device (5) adapted to be coupled to the housing, the delivery device comprising a reservoir (760) adapted to contain a fluid drug, and an expelling assembly (300) adapted for cooperation with the reservoir to expel fluid drug out of the reservoir and through the skin of the subject via the cannula when the delivery device has been coupled to the housing.

27. A medical device as in claim 1, wherein the cannula is in the form of a sensor device.

28. An assembly comprising a medical device as in claim 1 and a process unit adapted to be coupled to the housing, wherein the cannula is in the form of a sensor device and the process unit is adapted to transmit and/or process data acquired via the sensor.

29. A method of inserting a cannula into the subcutaneous tissue of a subject, comprising the steps of:
providing a cannula having a distal end portion adapted to be arranged through the skin of the subject and having a distal opening, and a needle arranged coaxially with and being axially moveable relative to the cannula, the needle comprising a pointed distal end,
advancing the cannula with the distal end of the needle projecting there from through the dermis of the subject,
further advancing the cannula into the sub-cutis of the subject with the distal end of the cannula projecting relative to the needle.

30. A method as in claim 29, wherein the distal end of the needle is arranged short of the distal end of the cannula during advancement of the cannula into the sub-cutis.

31. A method as in claim 29, wherein the needle is arranged either within the cannula or the needle is hollow and arranged outside the cannula.

32. An assembly comprising a medical device (901) as in any of claims 1-15, 17 and 19-25, and a delivery device (902) adapted to be coupled to the housing (923), the delivery device comprising a reservoir adapted to contain a fluid drug, and an expelling assembly adapted for cooperation with the reservoir to expel fluid drug out of the reservoir and through the skin of the subject via the cannula (971) when the delivery device has been coupled to the housing, wherein the delivery device in a situation of use in which the medical device has been applied towards the skin of a subject covers the cannula in its extended position.

33. A medical device as in claim 1, further comprising a reservoir adapted to contain a fluid drug, and an expelling assembly adapted for cooperation with the reservoir to expel fluid drug out of the reservoir and through the skin of the subject via the cannula, wherein the medical device in a situation of use in which the medical device has been applied towards the skin of a subject covers the cannula in its extended position.

34. An assembly comprising:
a transcutaneous device unit adapted for application towards a skin surface of a subject and comprising a housing, and an extendable transcutaneous device having a distal end portion adapted to be arranged through the skin of the subject at an inclined angle relative to the skin surface, and
a process unit adapted to be releasably coupled to the housing, the process unit comprising a process assembly adapted for cooperation with the transcutaneous device, wherein the process unit in a situation of use in which the assembly has been applied towards the skin of a subject covers the cannula in its extended position, and wherein at least partial removal of the process unit from the transcutaneous device unit allows inspection of the introduction site of the transcutaneous device through the skin surface.

35. An assembly comprising:
a medical device comprising a housing adapted for application towards a skin surface of a subject, an insertable cannula having a distal end portion with a distal opening and being adapted to be arranged through the skin of the subject at an inclined angle relative to the skin surface, and
a delivery device (902) adapted to be releasably coupled to the housing (923), the delivery device comprising a reservoir adapted to contain a fluid drug, and an expelling assembly adapted for cooperation with the reservoir to expel fluid drug out of the reservoir and through the skin of the subject via the cannula (971) when the delivery device has been coupled to the housing, wherein the delivery device in a situation of use in which the medical device has been applied towards the skin of a subject covers the cannula in its extended position, and wherein at least partial removal of the delivery device from the medical device allows inspection of the introduction site of the cannula through the skin surface.
